(19) United States
(12) Patent Application Publication
(10) Pub. No.: US 2006/0148023 A1
(43) Pub. Date: Jul. 6, 2006

(54) Affinity labeling of serine proteases for simultaneous detection of multiple serine protease activity levels

(76) Inventors: David J. Phelps, Madison, WI (US); Gary L. Johnson, Bloomington, MN (US); Brian W. Lee, Minnetonka, MN (US); Zbigniew Darzynkiewicz, Chappaqua, NY (US); Jerzy Grabarek, Lodz (PL)

Correspondence Address: SCHWEGMAN, LUNDBERG, WOESSNER & KLUTH, P.A., P.O. BOX 2938, MINNEAPOLIS, MN, 55402, US

(21) Appl. No.: 10/872,824

(22) Filed: Jun. 21, 2004

Related U.S. Application Data
(63) Continuation of application No. PCT/US02/40689, filed on Dec. 19, 2002.
(60) Provisional application No. 60/342,704, filed on Dec. 21, 2001.

(51) Int. Cl. C12Q 1/37

(52) U.S. Cl. 435/23

(57) ABSTRACT

The invention provides assay methods and reagents useful for evaluating the level of enzyme activities within living cells. Enzyme activity levels within living cells, such as Serine proteases, can be key determinates in assessing; 1) the presence of tumor (cancer) cells, 2) the predictive efficacy of a chemotherapeutic treatment regimen using a particular therapeutic agent or process, 4) the probability of graft rejection or acceptance, and 5) the disease state status of a cell. The identification of the up or down regulation relationships of serine proteases within living cell systems, provides a rapid, yet finely tuned mechanism for predicting the current and future physiological state of these cell populations.

### RELATED APPLICATIONS

This application is a continuation under 35 U.S.C. 111 (a) of International Application No. PCT/US2002/040689 filed Dec. 19, 2002 and published in English as WO 2003/060466 A3 on Jul. 24, 2003, which claimed priority under 35 U.S.C. 119(e) from U.S. Provisional Application Ser. No. 60/342,704 filed Dec. 21, 2001, which applications and publication are incorporated by reference and made a part hereof.

### BACKGROUND OF THE INVENTION

Serine (Ser) proteases are active enzymes that contain Ser at the active center, which participates in the formation of an intermediate ester to transiently form an acyl-enzyme complex. The most characterized enzymes of this type are tryptases and chymases. Trypsin and chymotrypsin are the most well known examples of these types of proteases. Involvement of Ser proteases in apoptosis has been mostly studied by observing whether particular apoptotic events can be prevented by the specific inhibitors of these enzymes. In the early studies Gorczyca et al., (Gorczyca et al., Int J Oncol, 1992, 1:639-648) have shown that fragmentation of DNA in HL-60 cells treated with DNA topoisomerase inhibitors to induce apoptosis was prevented by irreversible inhibitors of Ser proteases such as diisopropylfluoro-phosphate (DFP), N-tosyl-L-phenylalanine chloromethyl ketone (TPCK) and N-tosyl-L-lysine chloromethyl ketone (TLCK), as well as by excess of the substrates N-tosyl-L-arginine methyl ester (TAME) and N-benzoyl-L-tyrosine ethyl ester (BTEE).

Concurrently, Bruno et al., (Bruno et al., Leukemia, 1992, 6:1113-1120; Bruno et al., Oncol. Res., 1992, 4:29-35) observed that the same inhibitors and substrates inhibited nuclear fragmentation as well as fragmentation of DNA in other cell types, including thymocytes treated with the corticosteroid prednisolone. It was also observed that these inhibitors prevented destabilization of double-stranded DNA (Hara et al., Exp Cell Res, 1996, 223:372-384), which during apoptosis becomes sensitive to denaturing agents and can be detected as single-stranded DNA (Hotz et al., Exp. Cell Res., 1992, 201:184-19 1). These initial observations were confirmed in many subsequent studies and in other cell systems (Hughes et al., Cell Death Differ., 1998, 5:1017-1027; Kim et al., Int. J. Oncol., 2001, 18:1227-1232; Ghibelli et al., FEBS Lett., 1995, 377:9-14; Lotem et al., Proc Natl Acad Sci USA, 1996, 93:12507-12; Mansat et al., FASEB J, 1997, /1:695-702; Gong et al., Cell Growth Differ, 1999, 10:491-502; Komatsu et al., J. Biochem (Tokyo), 1998, 124:1038-44; Yoshida et al., Leukemia, 1996, 10:821-4; Weaver et al., Biochem Cell Biol, 7/:488-500; Park et al., Cytokine, 2001, 15:166-70). It should be noted, however, that while serine protease inhibitors prevent nuclear and DNA fragmentation triggered by different inducers, they themselves, especially after prolonged cell exposure, induce cell death that resembles apoptosis (Hara et al., Exp Cell Res, 1996, 223:372-384; Lu et al., Arch Biochem Biophys, 1996, 334:175-81).

The best recognized apoptosis-specific Ser proteases are granzymes A and B which are abundant in granules of cytotoxic T lymphocytes (CTL) and natural killer (NK) cells (Zapata et al., J. Biol. Chem., 1998, 273:6916-6920; Wright et al., Biochem. Biophys. Res. Commtin., 1998, 245:797-803; Shi et al., J Exp Med, 1992, 176:1521-9; Kam et al., Biochim Biophys Acta, 2000, /477:307-23; Jans et al., J Cell Sci, 1998, 111:2645-54; Estabanez-Perpina et al., J Biol Chem, 2000, 321:1203-1214). Granzyme B can cleave procaspase-3, -6, -7, -8, -9, and -10, and most likely, it activates endogenous caspases of the lymphocyte-target cells, thereby inducing their apoptosis (Zapata et al., J. Biol. Chem., 1998, 273:6916-6920). Granzyme A appears not to be associated with activation of caspases and it cleaves proteins independently of the latter (Shi et al., J Exp Med, 1992, 176:1521-9; Kam et al., Biochim Biophys Acta, 2000, 1477:307-23). Since granzymes A and B were studied predominantly in CTL or NK cells, it is unknown whether they play any role in apoptosis of other cell types.

Another apoptotic Ser protease is the 24-kD enzyme (AP24) shown to have the capacity to activate internucleosomal DNA fragmentation (Wright et al., J Exp Med, 1997, 186:1107-17; Wright et al., Cancer Res, 1998, 58:5570-6). Other Ser proteases that may function during apoptosis are the nuclear matrix-associated histone H1 specific enzyme induced by DNA damage (Kutsyi et al., Radiat Res, 1994, 140:224-229), the protease activated by Ca²⁺ (Zhivotovsky et al., Biochem Biophys Res Commun, 1997, 233:96-101) and myeloblastin (Bories et al., Cell, 1989, 59:959-968). Most recently a new Ser protease, HtrA2/Omni, that is released from the mitochondria and interacts with the caspase inhibitor XIAP in a similar way as Smac/Diablo promoting cell death, has been identified (Suzuki et al., Molecular Cell, 2001, 8:613-621, Verhagen et al., J Biol Chem, 2001, 277:445-454, Martins et al., J Biol Chem, 2001, 277:439-444. It is unknown whether the Ser cathepsins A and G are involved in apoptosis although the cysteine cathepsin B and aspartate cathepsin D are present in lysosomes and endosomes and they may participate in heterophagic degradation of apoptotic bodies (Johnson et al., Leukemia, 2000, 14:1695-1703, Leist et al., Nature Rev Mol Cell Biol, 2001, 2:589-598).

Ser proteases also play an important role as markers of tumor malignancy. For example, several Ser proteases have been identified in prostate cells and their enzymatic activity has been shown to have a positive correlation with the development of prostate cancer as well as the degree of tumor malignancy (Yousef et al., J Biol Chem 2001, 276:53-61, Chen et al., J Biol Chem 2001, 276:21434-42, Takayama et al., Biochemistry, 2001, 40:1679-87, Magee et al., Cancer Res., 2001, 61:5692-6). Ser protease activity is also a diagnostic and prognostic marker in other tumors, such as breast carcinoma (Ulutin & Pak, Radiat Med 2000, 18:273-6,Yousef et al., Genomics, 2000, 69:331-41), and carcinomas of the head and neck (Lang et al., Br. J Cancer 2001, 84:237-43).

Activities of Ser proteases are also altered in a variety of other diseases. As mentioned, the Set protease, granzyme B, is the key enzyme that is activated in a variety of cell-mediated immunological reactions. These cell-mediated responses include rejection of transplanted tissue (organs) and infections (Zapata et al., J. Biol. Chem., 1998, 273:6916-6920; Wright et al., Biochem. Biophys. Res. Commun., 1998, 245:797-803; Shi et al., J Exp Med, 1992, 176:1521-9; Kam et al., Biochim Biophys Acta, 2000, 1477:307-23; Jans et al., J Cell Sci, 1998, 111:2645-54; Estabanez-Perpina et al., J Biol Chem, 2000, 321:1203-1214).

There is currently a need for novel assay methods for measuring the activity or presence of more than one Ser protease. Such methods would facilitate the study of the role of Ser proteases in the altered status of living cells, as demonstrated by diseases such as Alzheimers, cancer, autoimmune reactions as well as other apoptotic and disease states. Such methods could also be utilized in the development of diagnostic methods that would specifically identify the aforementioned diseases as well as numerous others.

### SUMMARY OF THE INVENTION

Applicant has discovered that two or more serine protease activity levels can be measured in one or more living cells. Thus, the invention provides a method for determining the activity levels of two or more Ser proteases in one or more viable whole cells, comprising: 1) contacting the cells with two or more serine protease affinity labeling agents; and 2) detecting the presence of each affinity labeling agent in the cells; wherein the presence and relative abundance of each serine protease affinity labeling agent correlates with the respective serine protease activity levels of the cells.

The invention also provides:

assay reagents comprising at least 2 serine protease affinity labeling agents with different labels; and a suitable carrier;

a method for detecting and/or predicting the rejection of tissue or organ transplant wherein the presence or relative abundance of the group L detector molecule within the patient lymphocytes (“natural killer”; NK cells) or in cells of the transplanted organ (tissue) differs prior to- or at the time- of rejection from non-stimulated or pre-transplant tissue, by: 1) contacting the respective NK or organ tissue) cells with at least two serine protease affinity labeling agents; and 2) detecting the presence or relative abundance of each agent, wherein the presence or relative abundance of each agent is predictive of the tissue rejection response or NK cell activation;

a method for diagnosis and prognostic assessment of other cell-mediated immunological reactions, wherein the presence or relative abundance of each affinity labeling agent is characteristic of a particular type of cell mediated immunological reaction by; 1) contacting the cells with at least two serine protease affinity labeling agents and 2) detecting the presence or relative abundance of each agent in the cells, wherein the presence or relative abundance of each agent correlates with the detection and severity of the disease.

The invention provides methods which are useful for screening compounds, including libraries of chemical compounds, to identify therapeutic agents that modulate serine protease activity. The methods of the invention can be used to identify agents which induce, or reduce or inhibit apoptosis, as well as to identify therapeutic agents that are useful to treat diseases that are associated with serine protease activity. Techniques for screening chemical libraries are known in the art, and can be adapted for use in the methods described herein.

### DETAILED DESCRIPTION

The following definitions are used, unless otherwise described. “Red” is a fluorescent dye such as a rhodamine, BODIPY, Cy dye, etc. which is excited by light >520 nm. “Green” is a fluorescent dye such as fluorescein, BODIPY FL or Cy-2 etc, which is excited around 488 nm. “Cold” refers to a group that does not fluoresce, is not colored, is not radioactive and which is not normally considered a hapten. Examples of “cold” groups include, but are not limited to tosyl and carbobenzyloxy (Z). Halo is fluoro, chloro, bromo, or iodo. Alkyl, denotes both straight and branched groups; but reference to an individual radical such as “propyl” embraces only the straight chain radical, a branched chain isomer such as “isopropyl” being specifically referred to. Aryl denotes a phenyl radical or an ortho-fused bicyclic carbocyclic radical having about nine to ten ring atoms in which at least one ring is aromatic. Heteroaryl encompasses a radical attached via a ring carbon of a monocyclic aromatic ring containing 4 to 9 ring atoms consisting of carbon and one to four heteroatoms each selected from the group consisting of non-peroxide oxygen, sulfur, and N(X) wherein X is absent or is H, O, (C₁-C₄)alkyl, phenyl or benzyl, as well as a radical of an ortho-fused bicyclic heterocycle of about eight to ten ring atoms derived therefrom, particularly a benz-derivative or one derived by fusing a dimethylene, trimethylene, or tetramethylene diradical thereto.

The term “serine protease affinity labeling agent ” includes any agent capable of selectively binding, in a covalent manner, to one or more active serine proteases and facilitating their detection by analytical means. Accordingly, such an agent can include a fluorescent label, a radioactive label, or a hapten, or biotin as described herein. For example, one serine protease affinity labeling agent that can be used in the methods of the invention is a compound of formula I:
L-A-X—NH—CH(R′)C(═O)CH₂Cl (I)
wherein:

L is a detectable group;

A is a direct bond or a linker;

X is absent, an amino acid, or a peptide;

R′ is hydrogen or (C₁-C₆)alkyl, wherein the alkyl is optionally substituted with one or more, (1, 2, 3, or 4) substituents independently selected from the group consisting of guanidino, —C(═O)NRₐR_{b}, —C(═O)OR_{c}, halo, —NRₐR_{b}, aryl, heteroaryl, —OR_{c}, or —SR_{c};

each Rₐ and R_{b} is independently hydrogen, (C₁-C₆)alkyl, phenyl, benzyl, or phenethyl; or Rₐ and R_{b} together with the nitrogen to which they are attached form a pyrrolidino, morpholino, or thiomorpholino ring; and

each R_{c} is independently hydrogen, (C₁-C₆)alkyl, phenyl, benzyl, or phenethyl;

wherein any aryl or heteroaryl is optionally substituted with one or more (e.g. 1, 2, 3, or 4) substituents independently, selected from the group consisting of halo, nitro, cyano, hydroxy, mercapto, (C₁-C₆)alkyl, (C₁-C₆)alkoxy, trifluoromethyl, or trifluoromethoxy;

or a salt thereof.

For a compound of formula (I) L can preferably be a fluorescent label, a colored label, a radioactive label or hapten, or biotin; more preferably, L can be a fluorescent label (e.g. 5(6)-carboxyfluorescein, sulforhodamine B), or a colored label (e.g. 4-nitrophenyl or 2,4-dintrophenyl), or biotin. For a compound of formula (I) X can preferably be a peptide having about 2 to about 10 amino acids; more preferably, X can be a peptide having about 2 to about 5 amino acids. The amino acid composition of peptide X will define the enzyme selectivity of the affinity label. Enzymes will frequently target a 1 to 10 amino acid sequence identifying hydrophilic and hydrophobic residues within the sequence via binding sites within the enzyme catalytic region. By selectively defining the composition of the peptide sequence, it has been shown that the target specificity of the enzyme substrate can be changed (Melo et al. Analytical Biochem, 2001, 293:71-77).

Specifically, L is a fluorescent label, a colored label, a radioactive label, biotin or a hapten.

More specifically, L is a fluorescent label or biotin.

Preferably, L is 5(6)-carboxyfluorescein, or sulforhodamine B.

Specifically, X is a peptide containing from 2 to 10 amino acids.

More specifically, X is a peptide having about 2 to 5 amino acids.

Preferably, X is an amino acid sequence consisting of: phenylalanine-proline (FP), phenylalanine-arginine (FR), isoleucine-alanine-methionine (IAM), alanine-alanine (AA), valine-proline (VP), glutamic acid-glycine (EG) or alanine-alanine-proline (AAP) dimers and trimers of glycine and alanine (GG, GGG, AA, and AAA), and dimers and trimers of a mixture of these amino acids (GA, GAA, GGA, GAG, AGG, AGA, AAG and AG), (single letter abbreviations used are as follows; Ala (A), Arg (R), Asn (N), Asp (D), Cys (C), Glu (E), Gln (Q), Gly (G), His (H), lie (I), Leu (L), Lys (K), Met (M), Phe (F), Pro (P), Ser (S), Thr (T), Trp (W), Tyr (Y), and Val (V)).

For a compound of formula (I) X can preferably be a natural amino acid (e.g. alanine, glutamic acid, valine); more preferably, X is absent.

For a compound of formula (I) R′ can preferably be benzyl, 2-methylpropyl, 1-methylpropyl, 4-aminobutyl, or propylguanidino (arginine).

A preferred group of compounds of formula (I) are compounds wherein L is 5(6)-carboxyfluorescein, sulforhodamine B, or biotin; and R′ is benzyl, 2-methylpropyl, 1-methylpropyl, 4-aminobutyl, or propylguanidino (arginine).

A preferred compound of formula (I) is 5(6)-carboxyfluoresceinyl-L-phenylalanylchloromethyl ketone, 5(6)-carboxyfluoresceinyl-L-leucylchloromethyl ketone, or α-5(6)-carboxyfluoresceinyl-L-lysylchloromethyl ketone; or a salt thereof. Other preferred compound groups of this invention would include xanthene derivatives, such as fluorescein-5 or 6-isothiocyanate (FITC); rhodamines, such as rhodamine 110 and tetramethylrhodamine; and sulforhodamine labeled formulations, such as sulforhodamine 101 of the same phenylalanyl, leucyl, or lysyl chloromethyl ketone compounds.

For example, such agents may include fluorescent labels (e.g. fluorescein derivatives, sulforhodamine derivatives, Cy dye derivatives, BODIPY derivatives, coumarin derivatives, or any fluorescent dye that can be attached to an amino group directly or by linkers), colored labels (e.g. 4-nitrophenyl or 2,4-dintrophenyl, or any colored label that can be attached to an amino group directly or by linkers), a radioactive label (e.g. tritium, carbon-14 phosphorus-32), or biotin, or a hapten (e.g. digoxigenin, and dinitrophenyl), or the like. Other labels such as biotin and the various high affinity binding type hapten groups (digoxigenin, and dinitrophenyl) can be coupled to the affinity ligands to allow for the use of enzyme reporter group signal amplification. Commonly used enzymes include horseradish peroxidase (HRP), alkaline phosphatase (AP), β-galactosidase (BG), and urease (U). When coupled to avidin or IgG, for use in an avidin-biotin or hapten system respectively, the aforementioned enzyme molecules can convert colorless enzyme substrates to products with a detectable readout. The most commonly used chromogenic substrates include tetramethylbenzidine (TMB) for use with HRP labels, and nitro blue tetrazolium/5-bromo-4-chloro-3-indolyl phosphate (NBT/BCIP) for use with AP labels. Commercial chemiluminescent substrates of these enzymes can also be used. Radioactive labels, such as tritium, carbon-14, and phosphorous-32 can be used as a direct label or can also be coupled to avidin or anti-hapten IgG for radioactive detection.

There are two main classes of α-amino acids: “natural” and “unnatural” α-amino acids. Additionally there are a wide variety of β-amino acids, homologues of amino acids and molecules that mimic amino acids, such as isosteres.

“Natural amino acids” refers to the naturally occurring α-amino acid molecules typically found in proteins. These are: glycine, alanine, valine, leucine, isoleucine, serine, methionine, threonine, phenylalanine, tyrosine, tryptophan, cysteine, proline, histidine, aspartic acid, asparagine, glutamic acid, glutamine, arginine, and lysine.

“Natural amino acids” also exist in nature, which are not typically incorporated into naturally occurring proteins. Examples of these amino acids are: ornithine, γ-carboxyglutamic acid,hydroxylysine, citrulline, kynurenine, 5-hydroxytryptophan, norleucine, norvaline, hydroxyproline, phenylglycine, sarcosine, γ-aminobutyric acid and many others.

“Unnatural amino acids” are defined as those amino acids that are not found in nature and may be obtained by synthetic means well known to those schooled in amino acid and peptide synthesis. Examples of this class, which numbers in the many thousands of known molecules include: (t-butyl)glycine, hexafluoro-valine, hexafluoroleucine, trifluoroalanine, β-thienylalanine isomers, β-pyridylalanine isomers, ring substituted aromatic amino acids, at the ortho, meta, or para position of the phenyl moiety with one or more of standard groups of organic chemistry such as: fluoro-, chloro-, bromo-, iodo-, hydroxy-, methoxy-, amino-, nitro-, alkyl-, alkenyl-, alkynyl-, thio-, aryl-, heteroaryl- and the like.

It will be appreciated that amino acids and peptides can exist in L- or D-forms (enantiomers) and that certain amino acids with more than one chiral center, such as threonine, may exist in diastereomeric form. Further, when linked together in peptide chains, a mixture of L- and D-amino acids may be chosen to confer desired properties known in the art. Therefore, enantiomers, diastereomers and mixtures of these types are included in the claims.

Further, unnatural amino acids may exhibit other types of isomerism, such as positional and geometrical isomerism. These types of isomerism, coupled with or independent of optical isomerism, are also included in these claims.

In a specific preferred embodiment, the term “amino acid,” comprises the residues of the natural amino acids (e.g. Ala (A), Arg (R), Asn (N), Asp (D), Cys (C), Glu (E), Gln (Q), Gly (G), His (H), Hyl, Hyp, Ile (I), Leu (L), Lys (K), Met (M), Phe (F), Pro (P), Ser (S), Thr (T), Trp (W), Tyr (Y), and Val (V)) in D or L form, as well as amino acids which do not occur normally in proteins (e.g. phosphoserine, phosphothreonine, phosphotyrosine, hydroxyproline, gamma-carboxyglutamate, statine, penicillamine, omithine, citruline, β-methylalanine, and sarcosine). “Amino acid” also includes completely synthetic amino acids (e.g. α-propargyglycine, α-phenylglycine, α-t-butylglycine, homophenylalanine and all alpha amino acids synthesized by well known techniques). Also included are isosteres of amino acids and peptide bonds, well known in the art. When X is an amino acid in a compound of formula I, the amino terminus is on the left and the carboxy terminus is on the right.

The term “peptide” describes a sequence of 2 to 20 amino acids (e.g. as defined hereinabove) or peptidyl residues. Preferably a peptide comprises 2 to 10, or 2 to 5 amino acids. When X is a peptide in a compound of formula 1, the amino terminus is on the left and the carboxy terminus is on the right.

It will be appreciated that the methods of this invention can be used with all cell types that contain or express serine proteases. The cells may come from plant, bacteria or animal origins and may be from tissue samples, fluid samples or immortalized cell lines. Cells originating from animals include cells from; Protozoa, Mastigophora or Flagellata, Sarcodina, Sporozoa, Cnidospora and Ciliata; Porifera; Coelenterata; Platyhelminthes; Pseudocoelomates, Rotifera, Gastrotricha and Nematoda; Molluska; Annelida; Arthropoda; Bryozoa; Eichinodermata; Chordata; Hemichordata; Vertabrates, Fishes, Amphibians, Reptiles, Birds and Mammals. More specific, Mammalian cells include but are not limited to cells such as lypmhocytes, neutrophiles, mast cells, neutrophiles, basophilic leukocytes, eosinophilic leukocytes, erythrocytes, monocytes, osteoblasts, osteoclasts, neurons, astrocytes, oligodendricites, hepatocytes, squamous cells, macrophages, fibroblasts, endothelial cells, chondrocytes, granulocytes, karyocytes, spermatocytes, spermatozoa, and cells of Sertoli. Immortalized cell lines include but are not limited to HL-60, MCF-7, Jurkat, U937, Hela, and THP- 1.

The term “detectable group” includes any group that can be detected by analytical means. For example, suitable groups may be detectable by fluorescence spectroscopy, fluorescence microscopy, confocal fluorescence microscopy, fluorescence image analysis, flow cytometry, laser scanning cytometry, or plate multi-well fluorescence reader. Thus, suitable groups include fluorescent labels (e.g. fluorescein, rhodamines, Cy dyes, BODIPY dyes, sulforhodamine 101, phycobiliproteins, etc.). Other labels such as biotin and the various high affinity binding type hapten groups (digoxigenin and dinitrophenyl) can be coupled to the affinity ligands to allow for the use of enzyme reporter group signal amplification. Commonly used enzymes include horseradish peroxidase (HRP), alkaline phosphatase (AP), β-galactosidase (BG), and urease (U). When coupled to avidin or IgG, for use in an avidin-biotin or hapten system respectively, the aforementioned enzyme molecules can convert colorless enzyme substrates to colored readout product. Commercial chemiluminescent substrates of these enzymes can also be used. Radioactive labels, such as tritium, carbon-14, and phosphate-32 can be used as a direct label or can also be coupled to avidin or anti-hapten IgG for radioactive detection.

The nature of the “linker” is not critical provided the final compound of formula I has suitable properties (e.g. suitable solubility, cell toxicity, cell permeability, and ability to selectively react with the targeted serine protease group) for its intended application. The linker, denoted by the letter “A”, in the case where A can simply be a covalent bond, the detectable group (L) is attached directly to the N-terminal amino group of the peptide or amino acid e.g. amide linkage L-(C═O)—NH—R). A can also be any member of the class of linkers well known to those experienced in this field. Linkers are typically 4-18 atoms long, consisting of carbon, nitrogen, oxygen or sulfur atoms. Specific examples of linkers include ε-aminocaproic acid, di-ε-aminocaproic acid, oligomers of ethylene glycol (—O—(CH₂CH₂O)ₙCH₂CH₂— where n=0-5); or di- and triamines separated by 2 to 6 methylene groups, for example: —HN(CH₂)ₙ—NH(CH₂)ₘ—NH(CH₂)ₒ— where n, m and o are integers from 0 to 6. Typical linkers include ester (—OC(═O)—), thioester (SC(═O)—), thionoester (—OC(═S)—), carbonyl (—C(═O)—), and amide (—NHC(═O)— or —C(═O)NH—) groups, as well as divalent phenyl groups, and a 1 to 10 membered carbon chain, which chain can optionally comprise one or more double or triple bonds, and which chain can also optionally comprise one or more oxy (-O-) or thioxy (—S—) groups between carbon atoms of the chain. A preferred linker is a simple amide linkage ((—NHC(═O)—) facilitated by an activated carboxyl-N-hydroxysuccinimide leaving group or chemically similar coupling systems.

The assay reagents of the invention can also comprise one or more suitable carriers. Suitable carriers include polar, aprotic solvents (acetonitrile, DMSO and DMF); protic solvents (e.g. water, methanol, ethanol) or mixtures of polar, aprotic solvents and protic solvents.

The term “active serine protease” is defined as an active enzyme representative of a family of proteases which utilize serine as the electron exchange group. An “active serine protease” is an enzyme which is in its catalytically active configuration. Some examples of this type of enzyme include the known apoptosis-associated Ser proteases such as A24, granzymes A and B, Cathepsins A and G, HtrA2/Omni protease, as well as numerous yet unrecognized proteases that become activated during apoptosis. This term also includes other Ser proteases such as those associated with prostate tissue or cancer (prostate specific antigen (PSA), hepsin, prostasin, etc) and with other tissues and organs (such as elastase).

The term “necrosis” means the alternative, disorderly mode of cell death. Cells undergoing necrosis usually swell up and burst, releasing the cytoplasmic contents into the surrounding environment. Necrotic cell death does not require the energy derived from ATP.

The term “relative abundance” can be defined as; 1) the amount of fluorescent label observed in stimulated cells or tissue compared to the non-stimulated cells or tissue, 2) the ratio of one fluorescently labeled affinity ligand to the other fluorescently labeled affinity ligand in stimulated versus non-stimulated cells or tissue, 3) the amount of fluorescent label observed in disease state cells or tissue compared to normal/healthy cells or tissue, and 4) the ratio of one fluorescently labeled affinity ligand to the other fluorescently labeled affinity ligand in disease state cells or tissue versus normal/healthy cells or tissue.

Specific and preferred values listed below for radicals, substituents, and ranges, are for illustration only; they do not exclude other defined values or other values within defined ranges for the radicals and substituents.

Specifically, (C₁-C₆)alkyl can be methyl, ethyl, propyl, isopropyl, butyl, iso-butyl, sec-butyl, pentyl, 3-pentyl, or hexyl; (C₁-C₆)alkoxy can be methoxy, ethoxy, propoxy, isopropoxy, butoxy, iso-butoxy, sec-butoxy, pentyloxy, 3-pentyloxy, or hexyloxy; aryl can be phenyl, indenyl, or naphthyl; and heteroaryl can be furyl, imidazolyl, triazolyl, triazinyl, oxazoyl, isoxazoyl, thiazolyl, isothiazoyl, pyrazolyl, pyrrolyl, pyrazinyl, tetrazolyl, pyridyl, (or its N-oxide), thienyl, pyrimidinyl (or its N-oxide), indolyl, isoquinolyl (or its N-oxide) or quinolyl (or its N-oxide).

The assay reagents of the invention can also comprise one or more suitable carriers. Suitable carriers include DMSO, DMF, or other organic solvents which, when diluted out in aqueous buffer media, present minimal toxicity to the cell system being analyzed.

In cases where the affinity labels are sufficiently basic or acidic to form stable acid or base salts, use of the compounds as salts may be appropriate. Examples of such salts are organic acid addition salts formed with acids which form an acceptable anion, for example, tosylate, methanesulfonate, acetate, citrate, malonate, tartrate, succinate, benzoate, ascorbate, -ketoglutarate, and -glycerophosphate. Suitable inorganic salts may also be formed, including hydrohalide, sulfate, nitrate, bicarbonate, and carbonate salts.

Salts may be obtained using standard procedures well known in the art, for example by reacting a sufficiently basic compound such as an amine with a suitable acid affording an acceptable anion. Amines include, but are not limited to, ammonia, triethylamine, diphenylamine and other organic amines. Alkali metal (for example, sodium, potassium or lithium) or alkaline earth metal (for example calcium) salts of carboxylic acids can also be made.

### EXAMPLE 1

### General Protocol for Use of Multiple Serine Protease Affinity Labels

It can be inferred from the present data that 5(6)-Carboxyfluoresceinyl-L-phenylalanylchloromethyl ketone (FFCK) or Sulforhodaminyl-L-phenylalanylchloromethyl ketone (SFCK) and 5(6)-carboxyfluoresceinyl-L-leucylchloromethyl ketone (FLCK) or Sulforhodaminyl-L- leucylchloromethyl ketone (SLCK) do not bind to the active centers of the same enzymes and therefore it is possible that activation of two different serpases of the chymotrypsin-like family can be detected. FFCK and SFCK, having a Phe moiety, are expected to be specific inhibitors of chymotrypsin (EC 3.4.21.1). FLCK and SLCK, with a Leu moiety, should have preference to chymotrypsin C (EC 3.4.21.2) (Blow, D. M., Acc Chem Res, 1976, 9:145-152; Wilcox, P. E., Methods Enzymol, 1970, 19:64-108). The following is a description of how these reagents can be used for the simultaneous detection of chymotrypsin C (EC 3.4.21.2) and chymotrypsin C (EC 3.4.21.2).

Preparation of reagents: Fluorescent inhibitors of serine proteases (FLISP) reagents, 5(6)-Carboxyfluoresceinyl-L-phenylalanylchloromethyl ketone (FFCK), 5(6)-carboxyfluoresceinyl-L-leucylchloromethyl ketone (FLCK), Sulforhodaminyl 101 -L-phenylalanylchloromethyl ketone (SFCK) and Sulforhodaminyl 101 -L-leucylchloromethyl ketone (SLCK) are dissolved initially in dimethyl sulfoxide (DMSO; Sigma) to yield a 10 mM concentration. Aliquots are made from this stock solution and stored frozen at −20° C. protected from light. The reagent stock of the FLCK is then diluted directly into the cell culture media to give a 1× working reagent concentration of 20 μM and the reagent stock of the SFCK is then diluted directly into the cell culture media to give a 1× working reagent concentration of 5 μM. Dilution of the reagent into aqueous (cell culture) media is done just prior to cell exposure to preserve the labile chloromethyl ketone reactivity of FLISP reagent.

If they are to be used, unlabeled (cold) N-tosyl-phenylalanylchloromethyl ketone (TPCK) and N-tosyl-lysylchloromethyl keytone (TLCK) were obtained from Sigma Chemical Co.; concentrated solutions at 10 mM are freshly prepared in DMSO. Further dilutions are made in tissue culture media.

If they are to be used, the non-fluorescent poly-caspase inhibitor Z-VAD-FMK is obtained from Enzyme Systems Products. A 20 mM stock solution of Z-VAD-FMK is made in DMSO (Sigma) and the inhibitor was then diluted in culture media to obtain the final 50 μM concentration in the cultures.

Cells: Jurkat, HL-60 and U937 cells were obtained from American Type Culture Collection (ATCC; Rockville, Md.). They were cultured in 25 mL FALCON flasks (Becton Dickinson Co., Franklin Lakes, N.J.) using RPMI 1640 supplemented with 10% fetal calf serum, 100 units/mL penicillin, 100 mg/mL streptomycin and 2 mM L-glutamine (all from Gibco/BRL Life Technologies, Inc., Grand Island, N.Y.) in a humidified incubator set to maintain 37.5° C. and 5 % CO₂. At the onset of experiments, the cells were at an exponential and asynchronous growth phase with fewer than 5×10⁵ cells per/mL in culture. To induce apoptosis the cells were treated with 0.15 μM DNA topoisomerase I inhibitor camptothecin (CPT; Sigma Chemical Co., St. Louis, Mo.) for 3 hours.

Cell staining and evaluation by fluorescence plate reader: The Jurkat, HL-60 and U937 cells from the untreated or CPT treated cultures were centrifuged (200 g, 5 min) and resuspended in PBS at approximately 10⁶ cells per 5 ml volume. FLISP staining solutions were prepared by diluting 10 μL of a green 10 mM FLISP reagent stock solution (FLCK) and 2.5 μL of a red 10 mM FLISP reagent stock solution (SFCK) into 5 mL of culture medium yielding a final green FLISP concentration of 20 μM and a final red FLISP concentration of 5 μM. When detecting multiple serine proteases use different CMK labeled peptides i.e. FLCK and SFCK or FFCK and SLCK. The FLISP staining solution was removed by washing the cells in wash buffer (0.5% BSA in PBS +0.05% NaN₃, pH 7.4) and centrifuging for 5 minutes at 200 g. The washing step was repeated two more times with fresh wash buffer. After the final wash the cells were resuspended in 5 mL wash buffer and 100 μL per were added to a 96 well black microtiter plate (4 wells were used for each sample). Green and red fluorescence were measured simultaneously on a Spectra MAX Gemini XS fluorescence plate reader (Molecular Devices). Green cell fluorescence (FLCK) was then measured using a 488 nm excitation, 535 nm emission with a 515 nm cutoff. Red cell fluorescence (SFCK) was then measured using a 590 nm excitation, 620 emission with a 610 nm cutoff.

Results demonstrated that increases could be measured for each FLISP reagent in apoptotic cells when compared to non-apoptotic cells.

| | | FLCK Ratio | | SFCK Ratio |
|---|---|---|---|---|
| Cell Line | FLCK RFU | of I/NI | SFCK RFU | of I/NI |
| Jurkat I | 13.291 | 3.59 | 74.348 | 2.43 |
| Jurkat NI | 3.702 | | 30.596 | |
| HL-60 I | 7.552 | 1.59 | 49.045 | 1.28 |
| HL-60 NI | 4.748 | | 38.301 | |
| U937 I | 8.208 | 3.70 | 44.245 | 1.82 |
| U937 NI | 2.218 | | 24.341 | |

I is induced or apoptotic cells
NI is non-induced or non-apoptotic cells
All RFU values are an average of 4 wells.

From these data it is evident that these serine proteases have increased activity when the cells are induced into apoptosis. Chymotrypsin C (EC 3.4.21.2) shows a greater increase in activity levels than chymotrypsin (EC 3.4.21.1) for each cell type when measured simultaneously.

### EXAMPLE 2

### Synthesis of 5(6)-carboxyfluoresceinyl-L-phenylalanylchloromethyl ketone (FFCK)

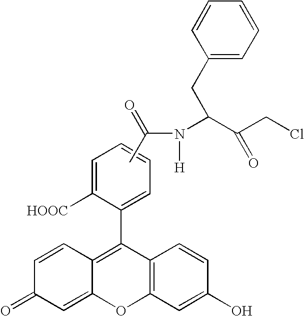

5-(and-6-)-Carboxyfluorescein, succinimidyl ester (80 mg, 0.17 mmole, FW 473.39, 5(6)-FAM) (Molecular Probes Inc., Eugene, Oreg., catalog number C-1311) was dissolved in 3 mL of dimethyl formamide (DMF). Phenylalanylchloromethyl ketone hydrochloride (40 mg, 0.17 mmole, FW 234) (Bachem Bioscience Inc., King of Prussia, Pa., catalog number N-1060) and diisopropylethyl amine (90 ul, Aldrich, Milwaukee, Wis.) were added to the solution. The reaction mixture was protected from light, stirred at room temperature for one hour and the solvent removed by rotary evaporation to provide an orange solid. The solid was partitioned between ethyl acetate and 10% aqueous hydrochloric acid (HCl), washed with 10% HCl and then water. The ethyl acetate fraction was dried over magnesium sulfate and the ethyl acetate removed by rotary evaporation to provide 35 mg dry weight, (37% yield) of 5(6)-carboxyfluoresceinyl-L-phenylalanylchloromethyl ketone (FFCK). Thin layer chromatography on silica gel (ethyl acetate: acetic acid, 97:3) gave a single spot of R_{f} 0.6.

### EXAMPLE 3

### Synthesis of 5(6)-carboxyfluorescyl-L-leucylchloromethyl ketone (FLCK)

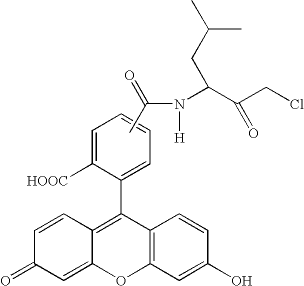

5-(and-6-)-Carboxyfluorescein, succinimidyl ester (82 mg, 0.17 mmole, FW 473.39, 5(6)-FAM) (Molecular Probes Inc., Eugene, Oreg., catalog number C-1311) was dissolved in 3 mL of dimethyl formamide (DMF). Leucylchloromethyl ketone ((35 mg, 0.17 mmole, FW 200.11) (Bachem Bioscience Inc., King of Prussia, Pa., catalog number N- 1 105) and diisopropylethyl amine (92 ul, Aldrich, Milwaukee, Wis.) were added to the solution. The reaction mixture was protected from light, stirred at room temperature for one hour and the solvent removed by rotary evaporation to provide an orange solid. The solid was partitioned between ethyl acetate and 15% aqueous hydrochloric acid (HCl), washed with 15% HCl and then water. The ethyl acetate fraction was dried over magnesium sulfate and the ethyl acetate removed by rotary evaporation to provide 72 mg dry weight, (81% yield) of 5(6)-carboxyfluoresceinyl-L-leucylchloromethyl ketone (FLCK). Thin layer chromatography on silica gel (ethyl acetate: acetic acid, 97:3) gave a single spot of R_{f} 0.7.

### EXAMPLE 4

Using procedures similar to those described herein, the following compounds of the formula (I) can also be prepared.

### α-5(6)-Carboxyfluoresceinyl-L-lysylchloromethyl ketone

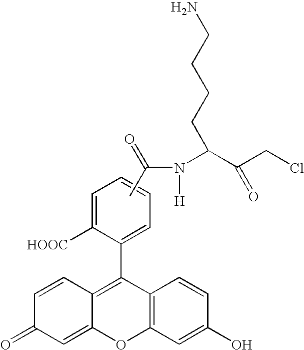

### 5(6)-Carboxyfluoresceinyl-L-arginylchloromethyl ketone

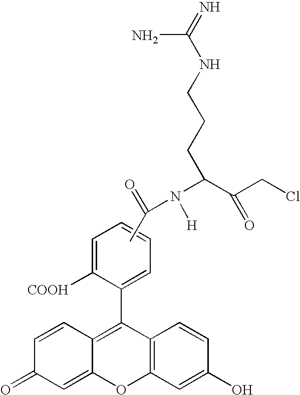

### Sulforhodaminyl-L-phenylalanylchloromethyl ketone

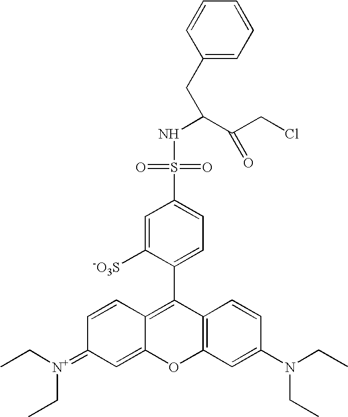

### Sulforhodaminyl-L-leucylchloromethyl ketone

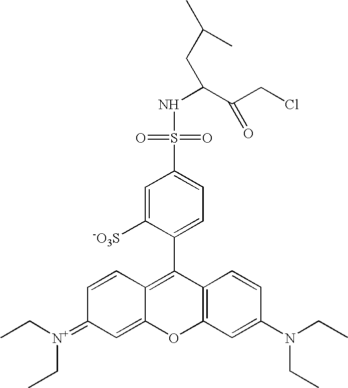

### α-Sulforhodaminyl-L-lysylchloromethyl ketone

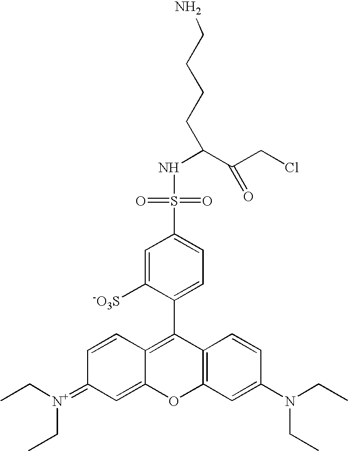

### Sulforhodaminyl-L-arginylchloromethyl ketone

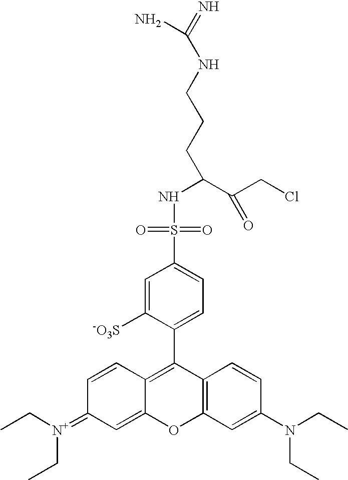

All publications, patents, and patent documents including 60/342,955, 60/342,778 and 60/342,704 are incorporated by reference herein, as though individually incorporated by reference. The invention has been described with reference to various specific and preferred embodiments and techniques. However, it should be understood that many variations and modifications may be made while remaining within the spirit and scope of the invention.

1. A method for determining multiple serine protease activity levels within one or more viable whole cells, comprising: 1) contacting the cells with at least two serine protease affinity labeling agent; and 2) detecting the presence of each affinity labeling agent in the cells; wherein the presence and abundance of each serine protease affinity labeling agent correlates with the serine protease composition activity of a separate serine protease within the cells.

2. The method of claim 1 wherein the cells are permeablized prior to contact with the agents.

3. The method of claim 1 wherein the presence of the first serine protease affinity label is detected concurrently with the detection of the presence of the second serine protease affinity label.

4. The method of claim 1 wherein the presence of the first serine protease affinity labeling agent is detected before or after the detection of the presence of the second serine protease affinity label.

5. The method of claim 1 wherein contacting the cells with the first serine protease affinity labeling agent is carried out concurrently with contacting the cells with the second serine protease affinity labeling agent.

6. The method of claim 1 wherein at least one serine protease affinity labeling agent is a compound of formula I:
L-A-X—NH—CH(R′)C(═O)CH₂Cl (I)
wherein:
L is a detectable group;
A is a direct bond or a linker;
X is absent, an amino acid, or a peptide;
R′ is hydrogen or (C₁-C₆)alkyl, wherein the alkyl is optionally substituted with one or more, (1, 2, 3, or 4) substituents independently selected from the group consisting of guanidino, —C(═O)NRₐR_{b}, —C(═O)OR_{c}, halo, —NRₐR_{b}, aryl, heteroaryl, —OR_{c}, or —SR_{c};
each Rₐ and R_{b} is independently hydrogen, (C₁-C₆)alkyl, phenyl, benzyl, or phenethyl; or Rₐ and R_{b} together with the nitrogen to which they are attached form a pyrrolidino, morpholino, or thiomorpholino ring; and
each R_{c} is independently hydrogen, (C₁-C₆)alkyl, phenyl, benzyl, or phenethyl;
wherein any aryl or heteroaryl is optionally substituted with one or more (e.g. 1, 2, 3, or 4) substituents independently, selected from the group consisting of halo, nitro, cyano, hydroxy, mercapto, (C₁-C₆)alkyl, (C₁-C₆)alkoxy, trifluoromethyl, or trifluoromethoxy;
or a salt thereof.

7. The method of claim 1 wherein at lease one serine protease affinity labeling agent is 5(6)-carboxyfluoresceinyl-L-phenylalanylchloromethyl ketone, 5(6)-carboxyfluoresceinyl-L-leucylchloromethyl ketone, α-5(6)-carboxyfluoresceinyl-L-lysylchloromethyl ketone, 5(6)-carboxyfluoresceinyl-L-arginylchloromethyl ketone, sulforhodaminyl-L-phenylalanylchloromethyl ketone, sulforhodaminyl-L-leucylchloromethyl ketone, α-sulforhodaminyl-L-lysylchloromethyl ketone, or sulforhodaminyl-L-arginylchloromethyl ketone; or a salt thereof.

8. The method of claim 1 wherein at least two serine protease affinity labeling agents are 5(6)-carboxyfluoresceinyl-L-phenylalanylchloromethyl ketone, 5(6)-carboxyfluoresceinyl-L-leucylchloromethyl ketone, α-5(6)-carboxyfluoresceinyl-L-lysylchloromethyl ketone, 5(6)-carboxyfluoresceinyl-L-arginylchloromethyl ketone, sulforhodaminyl-L-phenylalanylchloromethyl ketone, sulforhodaminyl-L-leucylchloromethyl ketone, α-sulforhodaminyl-L-lysylchloromethyl ketone, or sulforhodaminyl-L-arginylchloromethyl ketone; or a salt thereof.

9. A diagnostic method for determining the presence or absence of a disease characterized by the presence of two or more active serine proteases in one or more viable whole cells, comprising: 1) contacting the cells with a first serine protease affinity labeling agent and a second serine protease affinity labeling agent; and 2) detecting the presence of each affinity labeling agent in the cells; wherein the presence or relative abundance of the first serine protease affinity labeling agent and the presence or relative abundance of the second serine protease affinity labeling agent correlate with the presence or absence of the disease.

10. The method of claim 9 wherein the cells are permeablized prior to contact with the agents.

11. The method of claim 9 wherein the presence of the first serine protease affinity label is detected concurrently with the detection of the presence of the second serine protease affinity label.

12. The method of claim 9 wherein the presence of the first serine protease affinity labeling agent is detected before or after the detection of the presence of the second serine protease affinity label.

13. The method of claim 9 wherein contacting the cells with the first serine protease affinity labeling agent is carried out concurrently with contacting the cells with the second serine protease affinity labeling agent.

14. The method of claim 9 wherein contacting the cells with the first serine protease affinity labeling agent is carried out before or after contacting the cells with the second serine protease affinity labeling agent.

15. The method of claim 9 wherein the first serine protease affinity labeling agent is a compound of formula I as described in claim 6; or a salt thereof.

16. The method of claim 9 wherein the second serine protease affinity labeling agent is a compound of formula I as described in claim 6; or a salt thereof.

17. The method of claim 9 wherein the first serine protease affinity labeling agent is 5(6)-carboxyfluoresceinyl-L-phenylalanylchloromethyl ketone, 5(6)-carboxyfluoresceinyl-L-leucylchloromethyl ketone, α-5(6)-carboxyfluoresceinyl-L-lysylchloromethyl ketone, 5(6)-carboxyfluoresceinyl-L-arginylchloromethyl ketone, sulforhodaminyl-L-phenylalanylchloromethyl ketone, sulforhodaminyl-L-leucylchloromethyl ketone, α-sulforhodaminyl-L-lysylchloromethyl ketone, or sulforhodaminyl-L-arginylchloromethyl ketone; or a salt thereof.

18. The method of claim 17 wherein the second serine protease affinity labeling agent is 5(6)-carboxyfluoresceinyl-L-phenylalanylchloromethyl ketone, 5(6)-carboxyfluoresceinyl-L-leucylchloromethyl ketone, α-5(6)-carboxyfluoresceinyl-L-lysylchloromethyl ketone, 5(6)-carboxyfluoresceinyl-L-arginylchloromethyl ketone, sulforhodaminyl-L-phenylalanylchloromethyl ketone, sulforhodaminyl-L-leucylchloromethyl ketone, α-sulforhodaminyl-L-lysylchloromethyl ketone, or sulforhodaminyl-L-arginylchloromethyl ketone; or a salt thereof.

19. A method for determining whether a therapeutic agent increases serine protease activity in one or more viable whole cells, comprising: 1) contacting the cells with the therapeutic agent; 2) contacting the cells with a first serine protease affinity labeling agent and a second serine protease affinity labeling agent; and 3) detecting the presence of each of the affinity labeling agents in the cells; wherein the presence or relative abundance of the first serine protease affinity labeling agent and the presence or relative abundance of the second serine protease affinity labeling agent correlate with the ability of the therapeutic agent to increase different serine protease activity levels.

20. The method of claim 19 wherein the cells are contacted with the therapeutic agent before the cells are contacted with the affinity labeling agents.

21. The method of claim 19 wherein the cells are contacted with the therapeutic agent at the same time the cells is contacted with the affinity labeling agents.

22. The method of claim 19 wherein the cells are permeablized prior to contact with the agents.

23. The method of claim 19 wherein the presence of the first serine protease affinity label is detected concurrently with the detection of the presence of the second serine protease affinity label.

24. The method of claim 19 wherein the presence of the first serine protease affinity labeling agent is detected before or after the detection of the presence of the second serine protease affinity label.

25. The method of claim 19 wherein contacting the cells with the first serine protease affinity labeling agent is carried out concurrently with contacting the cells with the second serine protease affinity labeling agent.

26. The method of claim 19 wherein contacting the cells with the first serine protease affinity labeling agent is carried out before or after contacting the cells with the second serine protease affinity labeling agent.

27. The method of claim 19 wherein the first serine protease affinity labeling agent is a compound of formula I as described in claim 6; or a salt thereof.

28. The method of claim 27 wherein the second serine protease affinity labeling agent is a compound of formula I as described in claim 6; or a salt thereof.

29. The method of claim 19 wherein the first serine protease affinity labeling agent is 5(6)-carboxyfluoresceinyl-L-phenylalanylchloromethyl ketone, 5(6)-carboxyfluoresceinyl-L-leucylchloromethyl ketone, α-5(6)-carboxyfluoresceinyl-L-lysylchloromethyl ketone, 5(6)-carboxyfluoresceinyl-L-arginylchloromethyl ketone, sulforhodaminyl-L-phenylalanylchloromethyl ketone, sulforhodaminyl-L-leucylchloromethyl ketone, α-sulforhodaminyl-L-lysylchloromethyl ketone, or sulforhodaminyl-L-arginylchloromethyl ketone; or a salt thereof.

30. The method of claim 29 wherein the second serine protease affinity labeling agent is 5(6)-carboxyfluoresceinyl-L-phenylalanylchloromethyl ketone, 5(6)-carboxyfluoresceinyl-L-leucylchloromethyl ketone, α-5(6)-carboxyfluoresceinyl-L-lysylchloromethyl ketone, 5(6)-carboxyfluoresceinyl-L-arginylchloromethyl ketone, sulforhodaminyl-L-phenylalanylchloromethyl ketone, sulforhodaminyl-L-leucylchloromethyl ketone, α-sulforhodaminyl-L-lysylchloromethyl ketone, or sulforhodaminyl-L-arginylchloromethyl ketone; or a salt thereof.

31. A method for determining whether a therapeutic agent inhibits or reduces serine protease activity in one or more viable whole cells, comprising: 1) contacting the cells with the therapeutic agent; 2) contacting the cells with a first serine protease affinity labeling agent and a second serine protease affinity labeling agent; and 3) detecting the presence of each of the affinity labeling agents in the cells; wherein the presence or relative abundance of the first serine protease affinity labeling agent and the presence or relative abundance of the second serine protease affinity labeling agent correlate with the ability of the therapeutic agent to inhibit or reduce different serine protease activity levels.

32. The method of claim 31 wherein the cells are contacted with the therapeutic agent before the cells are contacted with the affinity labeling agents.

33. The method of claim 31 wherein the cells are contacted with the therapeutic agent at the same time the cells is contacted with the affinity labeling agents.

34. The method of claim 31 wherein the cells are permeablized prior to contact with the agents.

35. The method of claim 31 wherein the presence of the first serine protease affinity label is detected concurrently with the detection of the presence of the second serine protease affinity label.

36. The method of claim 31 wherein the presence of the first serine protease affinity labeling agent is detected before or after the detection of the presence of the second serine protease affinity label.

37. The method of claim 31 wherein contacting the cells with the first serine protease affinity labeling agent is carried out concurrently with contacting the cells with the second serine protease affinity labeling agent.

38. The method of claim 31 wherein contacting the cells with the first serine protease affinity labeling agent is carried out before or after contacting the cells with the second serine protease affinity labeling agent.

39. The method of claim 31 wherein the first serine protease affinity labeling agent is a compound of formula I as described in claim 6; or a salt thereof.

40. The method of claim 39 wherein the second serine protease affinity labeling agent is a compound of formula I as described in claim 6; or a salt thereof.

41. The method of claim 31 wherein the first serine protease affinity labeling agent is 5(6)-carboxyfluoresceinyl-L-phenylalanylchloromethyl ketone, 5(6)-carboxyfluoresceinyl-L-leucylchloromethyl ketone, α-5(6)-carboxyfluoresceinyl-L-lysylchloromethyl ketone, 5(6)-carboxyfluoresceinyl-L-arginylchloromethyl ketone, sulforhodaminyl-L-phenylalanylchloromethyl ketone, sulforhodaminyl-L-leucylchloromethyl ketone, α-sulforhodaminyl-L-lysylchloromethyl ketone, or sulforhodaminyl-L-arginylchloromethyl ketone; or a salt thereof.

42. The method of claim 41 wherein the second serine protease affinity labeling agent is 5(6)-carboxyfluoresceinyl-L-phenylalanylchloromethyl ketone, 5(6)-carboxyfluoresceinyl-L-leucylchloromethyl ketone, α-5(6)-carboxyfluoresceinyl-L-lysylchloromethyl ketone, 5(6)-carboxyfluoresceinyl-L-arginylchloromethyl ketone, sulforhodaminyl-L-phenylalanylchloromethyl ketone, sulforhodaminyl-L-leucylchloromethyl ketone, α-sulforhodaminyl-L-lysylchloromethyl ketone, or sulforhodaminyl-L-arginylchloromethyl ketone; or a salt thereof.

43. The method of claim 1 wherein detection is carried out using a flow cytometer; a laser scanning cytometer; a fluorescence microplate reader; a chromogenic microplate reader; a fluorescence microscope; a confocal microscope; a bright-field microscope; or a high content scanning system; or PAGE and western blot analysis.

44. The method of claim 1 wherein the presence of the labeling agents is detected in the cells by lysing the cells to provide a cell lysate and then detecting the presence of the labeling agents in the cell lysate.

45. An assay kit comprising packaging materials comprising 1) at least two serine protease affinity labeling agents, 2) instructions for using the agents to determine the serine protease activity levels of one or more cells, 3) an optional wash buffer compatible with all types of affinity labeling reagents, 4) an optional fixative reagent for preserving the affinity labeled cells for later evaluation, and 5) optional serine protease cold (no detection label present) affinity labeling agents.

46. An assay kit comprising packaging materials comprising 1) at least two serine protease affinity labeling agents, 2) instructions for using the agents for determining the presence or absence of a disease characterized by the presence of one or more active serine proteases, 3) an optional wash buffer compatible with all types of affinity labeling reagents, 4) an optional fixative reagent for preserving the affinity labeled cells for later evaluation, and 5) optional serine protease cold (no detection label present) affinity labeling agents.

47. An assay kit comprising packaging materials comprising 1) at least two serine protease affinity labeling agents, 2) instructions for using the agents for determining whether a therapeutic agent increases serine protease activity in one or more viable whole cells, 3) an optional wash buffer compatible with all types of affinity labeling reagents, 4) an optional fixative reagent for preserving the affinity labeled cells for later evaluation, and 5) an optional serine protease cold (no detection label present) affinity labeling agents.

48. An assay kit comprising packaging materials comprising 1) at least two serine protease affinity labeling agents, 2) instructions for using the agents for determining whether a therapeutic agent reduces or inhibits serine protease activity, 3) an optional wash buffer compatible with all types of affinity labeling reagents, 4) an optional fixative reagent for preserving the affinity labeled cells for later evaluation, and 5) optional serine protease cold (no detection label present) affinity labeling agents.

49. The method of claim 1 wherein contacting the cells with a red-labeled serine protease affinity labeling agent is carried out concurrently with contacting the cells with a green-labeled serine protease affinity labeling agent, of different or same amino acid composition.

50. The method of claim 1 wherein contacting the cells with a green-labeled serine protease affinity labeling agent is carried out concurrently with contacting the cells with a red-labeled serine protease affinity labeling agent, of different or same amino acid composition.

51. The method of claim 1 wherein contacting the cells with a red-labeled serine protease affinity labeling agent is carried out concurrently with contacting the cells with a cold-labeled serine protease affinity labeling agent, of different or same amino acid composition.

52. The method of claim 1 wherein contacting the cells with a green-labeled serine protease affinity labeling agent is carried out concurrently with contacting the cells with a cold-labeled serine protease affinity labeling agent, of different or same amino acid composition.

53. The method of claim 1 wherein contacting the cells with a cold-labeled serine protease affinity labeling agent is carried out concurrently with contacting the cells with a green-labeled serine protease affinity labeling agent, of different or same amino acid composition.

54. The method of claim 1 wherein contacting the cells with a cold-labeled serine protease affinity labeling agent is carried out concurrently with contacting the cells with a red-labeled serine protease affinity labeling agent, of different or same amino acid composition.

55. The method of claim 1 wherein contacting the cells with a red-labeled serine protease affinity labeling agent is carried out concurrently with contacting the cells with a red-labeled serine protease affinity labeling agent, of different or same amino acid composition.

56. The method of claim 1 wherein contacting the cells with a green-labeled serine protease affinity labeling agent is carried out concurrently with contacting the cells with a green-labeled serine protease affinity labeling agent, of different or same amino acid composition.

57. A method for determining if one or more compounds within a chemical library modulate serine protease activity in a mammal comprising,
a) contacting a biological sample from the mammal with one or more compounds from the library, and
b) contacting the sample with a first serine protease affinity labeling agent and a second serine protease affinity labeling agent; and
c) detecting the level of the affinity labeling agents in the biological sample, and
d) comparing the level of affinity labeling agents in the biological sample with a control biological sample not exposed to the compound to determine whether the compound modulated the serine protease activity in the mammal, wherein the presence or relative abundance of the first serine protease affinity labeling agent and the presence or relative abundance of the second serine protease affinity labeling agent correlate with the ability of the compound to modulate serine protease activity.

58. A method for determining if one or more compounds within a chemical library induce apoptosis in mammalian cells comprising,
a) contacting the cells with one or more compounds from the library, and
b) contacting the cells with a first serine protease affinity labeling agent and a second serine protease activity labeling agent; and
c) detecting the level of the affinity labeling agents in the cells, and
d) comparing the level of affinity labeling agents in the cells with a control cell sample not exposed to the compound to determine whether the compound modulated the first serine protease activity and the second serine protease activity, wherein the presence or relative abundance of the first serine protease affinity labeling agent and the presence or relative abundance of the second serine protease affinity labeling agent correlate with the ability of the compound to induce apoptosis.

59. A method for determining if one or more compounds within a chemical library reduces or inhibits apoptosis in mammalian cells comprising,
a) contacting the cells with one or more compounds from the library, and
b) contacting the cells with a first serine protease affinity labeling agent and a second serine protease activity labeling agent; and
c) detecting the level of the affinity labeling agents in the cells, and
d) comparing the level of affinity labeling agents in the cells with a control cell sample not exposed to the compound to determine whether the level of the affinity labeling agents increased or decreased in the cells, wherein the presence or relative abundance of the first serine protease affinity labeling agent and the presence or relative abundance of the second serine protease affinity labeling agent correlate with the ability of the compound to reduce or inhibit apoptosis.

60. The method of claim 57, wherein at least one of the serine protease affinity labeling agents is a compound as described in claim 7.
